(19) United States
(12) Patent Application Publication
(10) Pub. No.: US 2021/0369457 A1
(43) Pub. Date: Dec. 2, 2021

(54) APPARATUS AND METHODS FOR IN-HEART VALVE SURGERY

(71) Applicant: THE TRUSTEES OF COLUMBIA UNIVERSITY IN THE CITY OF NEW YORK, New York, NY (US)

(72) Inventors: Vinayak BAPAT, New York, NY (US); Shalaka Vinayak BAPAT, New York, NY (US)

(21) Appl. No.: 17/402,301

(22) Filed: Aug. 13, 2021

Related U.S. Application Data
(63) Continuation of application No. PCT/US20/18342, filed on Feb. 14, 2020.
(60) Provisional application No. 62/806,462, filed on Feb. 15, 2019.

(51) Int. Cl. A61F 2/24

(52) U.S. Cl. CPC A61F 2/2445; A61F 2210/0014; A61F 2210/0076; A61F 2250/0024; A61F 2250/0039; A61F 2250/0019; A61F 2220/0016

(57) ABSTRACT

An annuloplasty ring is provided including a core defining a closed ring and comprising one or more flexible portions, wherein the core is capable of deformation about the flexible portion between a first configuration and a second configuration upon application of a predetermined force; a resilient intermediate layer; and a fabric cover layer.

### CROSS-REFERENCE TO RELATED APPLICATIONS

This application is a continuation of PCT/US2020/018342 filed Feb. 14, 2020, which claims benefit of priority to U.S. Provisional Application Ser. No. 62/806,462 filed Feb. 15, 2019, both of which are incorporated in their entirety herein.

### FIELD

The present disclosure relates to a new annuloplasty ring for use in heart Surgery, and more particularly to a novel device used in valve repair Surgery.

### BACKGROUND

Human heart valves, such as mitral and tricuspid valves, are sometimes damaged by disease or aging such that the valves no longer properly function. In such cases, heart valve surgery is often indicated. Although the valve can be replaced there is current emphasis on repairing the valve, as it has been shown to have positive impact on the heart function in addition to elimination of disadvantages imposed on patients such as need for anticoagulation.

In most valve repair operations, an annuloplasty ring or valvuloplasty ring is used in the repair of the damaged valve, in order to support the repair and avoid future dilatation of the annulus.

Mitral valve anatomy can influence the ring design. (FIG. 1) The mitral valve has two leaflets—anterior and posterior. The anterior leaflet is larger in area but is attached to 40% or less of the circumference, while the posterior leaflet is semilunar and shorter in height but is attached to the 60% of the circumference. The attachment of the leaflets to the heart muscle is referred to as ‘the annulus.’

The anterior annulus in relation to the anterior leaflet is made of fibrous tissue and is believed to resist dilatation while the posterior annulus is mainly comprised of muscular tissue and contributes to dilatation observed when the mitral valve is leaking/regurgitant.

Mitral valve regurgitation is a common entity observed in clinical practice. When surgery on the mitral valve is performed, abnormal leaflet tissue is resected, additional support elements called chordae are adjusted or implanted when necessary and finally the annulus is brought back to normal size and shape by suturing an annuloplasty ring to the atrial surface of mitral valve annulus. In certain pathologies and according to some surgeons only posterior annuloplasty is needed so as to correct the posterior annulus dilatation. Hence, some rings are designed as ‘incomplete rings’. Further, the advantage of the incomplete rings is they are easier to implant.

One ring to be used during conventional heart valve repair was the Carpentier-Edwards Classic annuloplasty ring for the mitral valve. It was used to provide support for the repaired native mitral annulus and to remodel the annulus into its proper shape and configuration after valve repair. To prevent annular dilatation in the future the Carpentier-Edwards Classic annuloplasty ring was made rigid in structure and was designed to encircle the entire native valve annulus, thus forming a nearly complete circumference at the annular level. The shape of the ring is designed to simulate the shape and configuration of a normal valve. Thus, the abnormally shaped valve, which has undergone repair, can be transformed into a valve with a normal shape and configuration through the incorporation of the annuloplasty device. It was secured to the native annulus by sutures that are placed through the native heart annulus and through the annuloplasty ring.

The drawback of this ring was the excess rigidity and uniplanar shape. Although it corrected the abnormality in the annulus and provided support, it did prevent the normal movement in the mitral annulus which occurs during cardiac cycle. Further, it needs to be sutured to the entire annulus. Proper exposure and suturing the ring to the anterior annulus can be a complicated procedure.

To address these issues various designs were made available. For example, semi-rigid complete rings encircled the entire mitral annulus and could have a uniplanar or multiplanar shape to mimic saddle shape of the mitral valve. But importantly, they were semi-rigid such that they allowed motion of the mitral valve annulus during the cardiac cycle. The semi-rigid property was achieved by using metal core of different properties or configuration. Examples of such rings are Physio1, Physio2, 3D Memo and Simulus rings.

Semi-rigid incomplete rings were similar in property as above but were incomplete and hence the anterior two ends did not meet each other and essentially they encircled only posterior annulus. Examples of such rings are CG future ring. Advantage of such ring was that these required to be sutured only to the posterior annulus.

Flexible complete rings/bands were constructed such that they were completely flexible and could adapt to any shape sutured. They provided less support than the rigid and semi-rigid rings.

Incomplete bands supported only the posterior annulus, again to prevent difficulty in suturing the ring to the entire annulus and also in certain circumstances where there was a possibility of systolic anterior motion resulting in left ventricular obstruction. These bands are commonly used during minimally invasive surgery.

Rigid rings of certain shape were constructed to reshape the annulus in a particular way to address a certain pathology. Many of these rings are rigid and encircled the entire annulus. Examples of these rings are, IMR Etiologix ring for ischemic mitral regurgitation, Geoform ring for ischemic mitral regurgitation and Myxo ring for myxomatous mitral disease.

The tricuspid valve anatomy is illustrated in FIG. 2. The tricuspid valve is made of three leaflets—anterior, posterior and septal. Similar to the mitral valve, a common pathology is leakage/regurgitation. This is usually secondary to the elevated pressure in the right side of the heart whereby the tricuspid annulus dilates, and the leaflets cannot meet each other in the center resulting in leak. When Tricuspid valve is repaired, incomplete and rigid rings are usually used.

There are few rings which are flexible in some portion and rigid in others. The main common theme in tricuspid rings is that they are typically incomplete. This is to avoid injury to the ‘conduction tissue’. Hence, the incomplete portion is placed to leave open a rea where the AV node resides. Damage to this results in heart block and requires a pacemaker.

Although valve repair is preferred over replacement, the durability of the repair depends on pathology and expertise of the surgeon. Hence, it is not uncommon for mitral regurgitation or sometimes mitral stenosis to develop and patient requiring second intervention. Until recently the second intervention possible was re-operation on the mitral valve and majority of the times the valve cannot be re-repaired but is replaced. Transcatheter heart valves (THV) which were initially manufactured to treat calcified stenosed aortic valve pathology, have now been used to treat failed mitral valve repairs. The idea behind this treatment is to perform valve replacement under X-ray and echocardiographic guidance within the mitral ring to avoid open heart surgery.

The results of the valve-in-ring (VIR) procedure have been mixed and not encouraging. This is because mitral rings vary in shape, rigidity and their completeness. For a VIR procedure to be successful and durable long term, the ring which is accommodating a THV inside it (1) must adapt a circular shape as otherwise it will deform the THV and (2) should provide anchor to the THV. Rigid rings cannot become circular and result in leakage around them and deform the THV function resulting in suboptimal result. Most semi rigid rings also never become fully circular. While rigid and semi rigid complete rings can provide anchoring to a TNV, flexible bands and incomplete rings (independent of their rigidity) cannot provide good anchor and result in embolization of the THV

Rigid complete rings and semi rigid complete rings are currently the most used rings for mitral valve repair and tricuspid valve repair. But for a VIR procedure they are still suboptimal due to their construction as they do not become fully circular and provide adequate support for the implanted THV.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

In one aspect, an annuloplasty ring is provided, including a core defining a closed ring and including one or more flexible portions, wherein the core is capable of deformation about the flexible portion between a first configuration and a second configuration upon application of a predetermined force; a resilient intermediate layer; and a fabric cover layer

In some embodiments, the flexible portions of the core include polymer. In some embodiments, the flexible portions of the core include a metallic coil. In some embodiments, the core includes a plurality of metal wires. The core can be fabricated of Elgiloy or Nitinol. In some embodiments, the core includes titanium. In some embodiments, the core includes PEEK.

In some embodiments, the intermediate layer includes polymer or rubber.

In another aspect, an annuloplasty ring is provided including a core defining a closed ring and including one or more weakened portions, wherein breakage of the weakened portions upon application of a predetermined force causes deformation between a first configuration and a second configuration; a resilient intermediate layer; and a fabric cover layer

In some embodiments, the weakened portions of the core include a smaller dimension than adjacent portions of the core. In some embodiments, the weakened portions of the core define a plurality of perforations therethrough. In some embodiments, the weakened portions of the core include a more brittle material than adjacent portions of the core. In some embodiments, the weakened portions of the core include a softer material than adjacent portions of the core.

In another aspect, an annuloplasty attachment for use with an annuloplasty ring is provided including a body portion defining end portions and a plurality of engagement members for securement to the annular tissue; and connection members disposed at each end portions of the body portion for connection with the end portions of an incomplete annuloplasty ring.

In some embodiments, the connection members are eyelets configured for suturing to the end portions of the incomplete annuloplasty ring. In some embodiments, the engagement members are barbs, tines or anchors.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic representation of a mitral valve of a human heart.

FIG. 2 is a schematic representation of a tricuspid valve of a human heart.

FIG. 3 is elevation view of an annuloplasty ring in accordance with an exemplary embodiment of the disclosed subject matter.

FIG. 4 is an enlarged view of the annuloplasty ring of FIG. 4 in partial cross-section.

FIG. 5 is an elevation view of an annuloplasty ring in accordance with another exemplary embodiment of the disclosed subject matter.

FIG. 6 is an enlarged cross-sectional view of an annuloplasty ring in accordance with a further embodiment of the disclosed subject matter.

FIG. 7 is an elevation view of an annuloplasty ring in accordance with another exemplary embodiment of the disclosed subject matter in a first configuration with the parts intact.

FIG. 8 is an elevation view of an annuloplasty ring in accordance with the exemplary embodiment of FIG. 7 in a second configuration with the parts separated.

FIG. 9 is elevation view of an annuloplasty attachment for attachment to an incomplete annuloplasty ring in accordance with an exemplary embodiment of the disclosed subject matter.

FIG. 10 illustrates the annuloplasty attachment of FIG. 9 attached to an incomplete annuloplasty ring disposed at the mitral valve of a human subject.

FIG. 11 illustrates the annuloplasty attachment of FIG. 9 attached to an incomplete annuloplasty ring disposed at the tricuspid valve of a human subject.

FIG. 12 is an elevation view of an annuloplasty attachment for attachment to an incomplete annuloplasty ring in accordance with another exemplary embodiment of the disclosed subject matter.

FIG. 13 is an elevation view of an annuloplasty attachment for attachment to an incomplete annuloplasty ring in accordance with a further exemplary embodiment of the disclosed subject matter.

FIG. 14 is an elevation view of an annuloplasty attachment pre-attached to an incomplete annuloplasty ring in accordance with a still further exemplary embodiment of the disclosed subject matter.

FIG. 15 is a cross-section view of the human heart, illustrating the annuloplasty attachment and an incomplete annuloplasty ring attached the heart.

FIG. 16 is a cross-section view of the human heart, illustrating an incomplete annuloplasty ring attached the heart and artificial replacement valve.

FIG. 17 is an elevation view illustrating an incomplete annuloplasty ring and an artificial replacement valve.

FIG. 18 is a cross-section view of the human heart, illustrating an annuloplasty attachment and an incomplete annuloplasty ring attached the heart along with an artificial replacement valve.

FIG. 19 is an elevation view illustrating an annuloplasty attachment and an incomplete annuloplasty ring along with an artificial replacement valve.

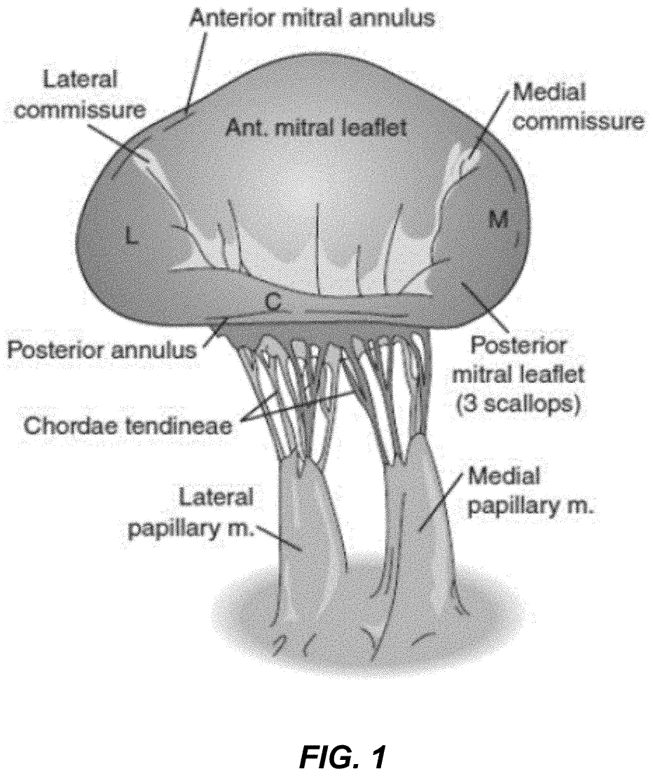

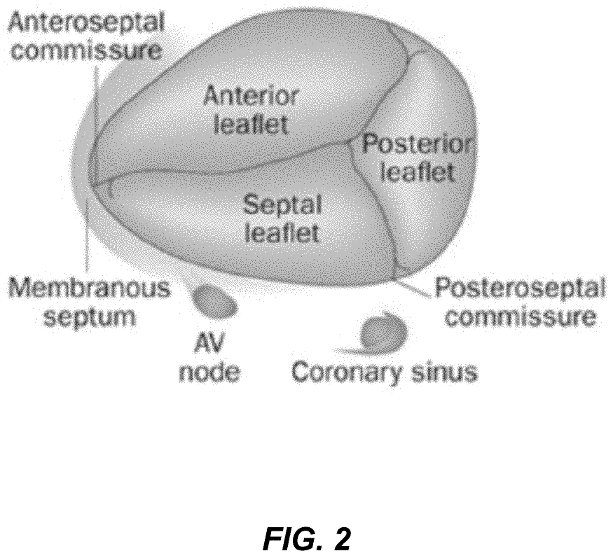

### DETAILED DESCRIPTION

Reference will now be made in detail to select embodiments of the disclosed subject matter, examples of which are illustrated in the accompanying drawings. The method and corresponding steps of the disclosed subject matter will be described in conjunction with the detailed description of the system.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

As used herein and in the appended claims, the singular forms “a,” “an,” and “the” include plural referents unless the context clearly dictates otherwise.

In accordance with the various embodiments of the disclosed subject matter, annuloplasty rings or devices for use in heart surgery are disclosed herein. More particularly, annuloplasty rings, devices and methods are provided for their use in heart valve repair surgery.

In some embodiments, the rings have rigid or semi rigid construction, and include a core fabricated of metal such as Elgiloy/Nitinol/Titanium and/or polymer such as PEEK, and can be easily molded in to any desired shape and will retain the shape when implanted during mitral and tricuspid repair.

When manufactured there may be high pressure sensitive points along the circumference of the ring which can allow a complete or partial break, and thus the ring adapting a circular shape. If the ring is made from an alloy, the joints used to secure the wire or wires can be either distributed to avoid overlap or made of a design such that it can be made circular.

The construction described herein allows the ring to be made of desired rigidity i.e., rigid or semi rigid, such that when it is manufactured and implanted, it can retain its desired first shape or configuration throughout the life cycle of the ring, thus, the ring will not change its shape or size after implantation. Moreover, if there is a need for valve-in-ring (VIR) procedure, the ring can be easily forced in to a second shape or configuration, e.g., a substantially circular shape, during a surgical procedure. Thus the design described herein provides good anchor by nature of its components, and also optimal shape of the transcatheter heart valve (THV) so as to achieve durable long term result.

The annuloplasty ring includes a core forming a closed ring, typically fabricated of metal such as Elgiloy, Nitinol or Titanium; an intermediate layer of resilient material, such as polymer or rubber. An outer layer is a fabric material applied over the intermediate layer of polymer or rubber. The core is configured to be deformed between a first configuration and a second configuration.

Rigidity is determined for the particular surgical need, and is property dependent on the nature of metal and its configuration. For example, in some embodiments, the core is fabricated from metal, e.g., titanium sheet or thick wire. Consequently, the ring will be rigid in structure and will maintain the shape irrespective of force applied to it from within. In other embodiments, the core is made of multiple thinner wires of Elgiloy or Nitinol. In this embodiment, the ring may become semi-rigid and allow a deformation to nearly circular shape.

In some embodiments, the core is made of a polymer such as PEEK. PEEK is known to be extremely rigid without increasing bulk. The PEEK material is molded in any shape and can be adapted to all existing rigid ring shapes. Annuloplasty rings in accordance with some embodiments, include a core fabricated from PEEK, which is covered by an intermediate layer of silastic or any other material similar to current rings, and then covered by an outer layer of fabric. The rigidity of the ring at the time of implant in its initial configuration remains unchanged. There are inherent areas of weakness or flexion which are incorporated within the PEEK structure. These will allow the ring to break/deform from a first configuration to a second configuration when a predetermined level of force is applied to the ring. The second configuration can be (a) substantially circular; (b) expand in size; or (c) provide an appropriate shape to anchor an implanted artificial valve.

In some embodiments, a semi rigid ring is described. According to the this embodiment, a combination of polymer and metal alloy can achieve semi rigidity to allow similar degree of ring motion with mitral valve motion as the current rings, The construction will allow the ring to assume circular shape after VIR. This is achieved as described below.

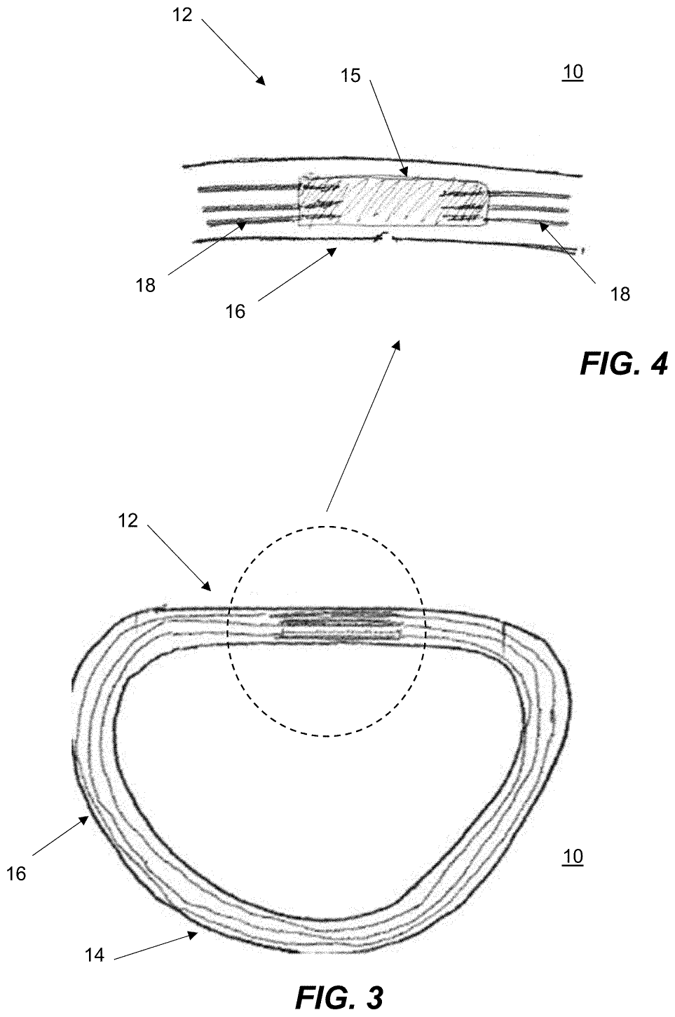

As illustrated in FIGS. 3 and 4, an embodiment of the ring 10 includes an anterior portion 12 and posterior portion 14. Ring 10, which includes a plurality of wires 18 in its construction, eliminates the anterior joint of conventional rings where wire forms are welded or joint together. FIG. 4 illustrates that the usual joint is replaced by polymer section 16 used to connect the wires 18. The polymer piece 16 is capable of controlled breakage by the surgeon when the applied force exceed a predetermined threshold. Upon application of the force, the ring 10 can assume a second configuration as described above. The ring 10 is covered with an outer layer 16 including polymer or rubber surrounded by fabric. The outer layers 16 serve two purposes, they provide a biocompatible and atraumatic covering from the core 18, and also following breakage of the polymer section 15 maintain the integrity of the closed ring shape.

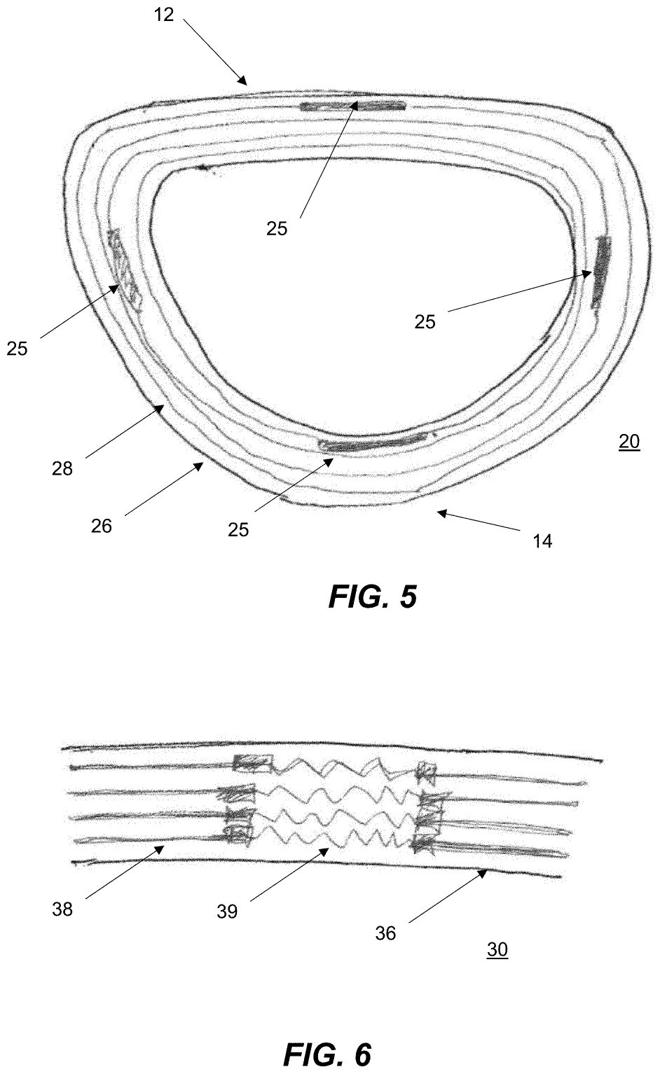

As illustrated in FIG. 5, another embodiment of the ring 20 include a core 28 having a plurality wires and a plurality of welding joints or polymer section 25 in various wire forms at different locations to avoid overlap of the welding points. This configuration allows the surgeon to impose circularity during VIR by applying a predetermined force to move the core 28 to its second configuration, e.g., a circular or expanded configuration. The ring 20 is covered with an outer layer 26 including polymer or rubber surrounded by fabric.

As illustrated in FIG. 6, a further embodiment of the ring 30 includes a core 38 having wires that are each joined with a coil 39. The configuration of ring 30 maintains a degree of semi rigidity when implanted in its initial configuration, but is capable of being molded to circular shape by application of a predetermined force by the surgeon. The ring 30 is covered with an outer layer 36 including polymer or rubber surrounded by fabric.

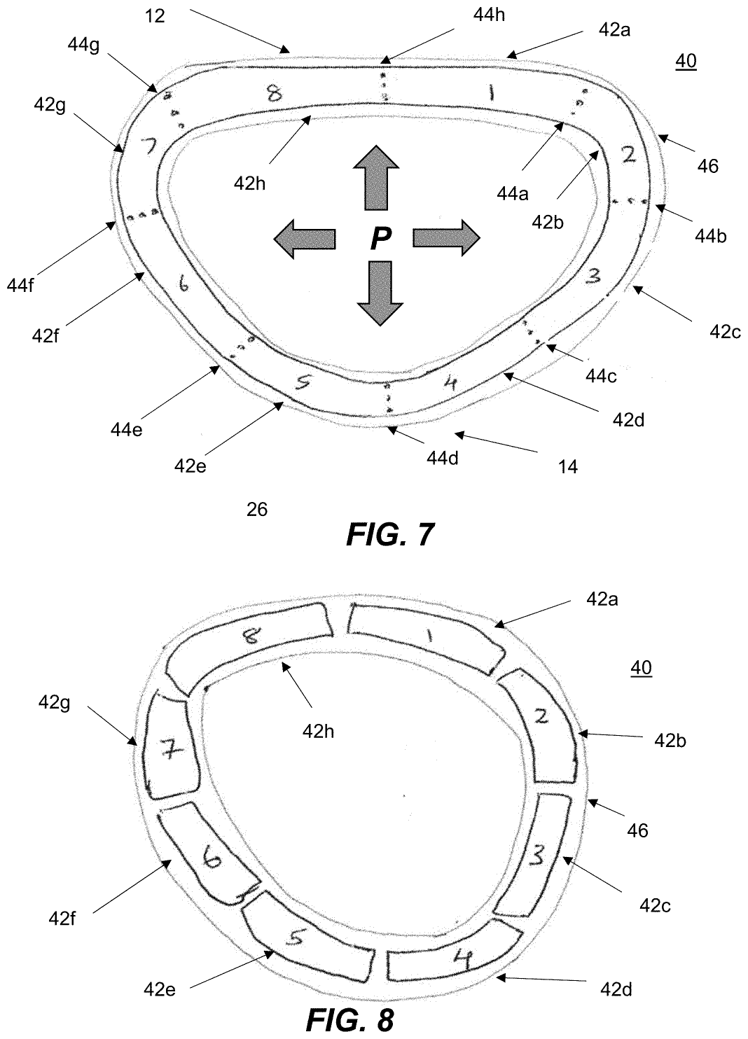

As illustrated in FIGS. 7 and 8, another embodiment of the ring 40 is described in which the core is constructed from a rigid polymer and includes predesigned weak spots 44a-44h. In some embodiments, the weakness is provided by e.g., including a plurality of perforation therethrough; by making the weak spots narrower or smaller than adjacent portions of the core, by using a more brittle or softer material for the weak spots than the adjacent core material, etc. When applies pressure from within exceeds a predetermined threshold (arrows P), the weak spots 44a-44h will break fully or partially, separating the ring into sections 42a-42h to make the ring circular. (FIG. 8) The ring 40 is covered with an outer layer 46 including polymer or rubber surrounded by fabric. As discussed above, the outer layers 46 help maintain the integrity of the closed ring shape after breakage of the weakened spots.

The same ring designs described herein will work in Tricuspid position as well with no change in functionality.

Another embodiment is disclosed herein is an attachment that is used to fill the gap in incomplete or broken rings in valve repair surgery. Most particularly, this is a novel attachment to be used in mitral valve and tricuspid valve repair surgery, whereby the attachment fills the gap between the two ends of an open/incomplete ring. The shape and function of the implanted ring is unaltered. However, by implanting this attachment, the assembly of the ring and the attachment will behave like a complete ring during a valve-in-ring (VIR) procedure.

The attachment described herein effectively converts an incomplete or partial ring on mitral and tricuspid side to a complete ring without the need of suturing and risk of altering shape of the annulus or damage to neighboring structure such as conduction tissue. The ring attachment is used as a separate attachment or as part of the incomplete band. The design could take one of the following embodiments.

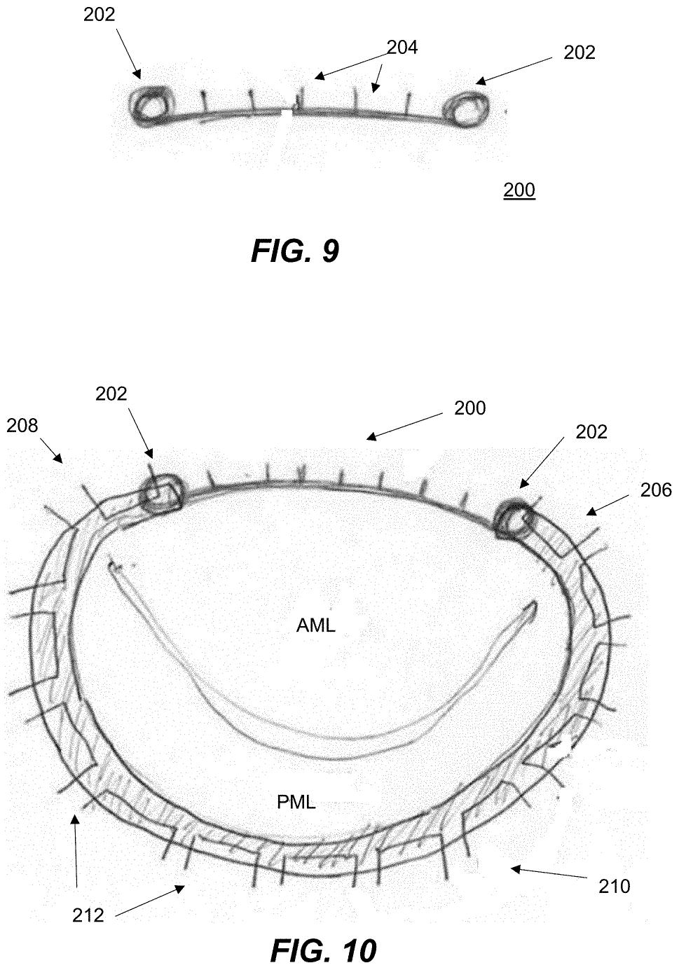

Attachment 200 is illustrated in FIG. 9, and is used to bridge the gap between the ends of an incomplete ring or band. As described herein, an incomplete ring or band refers to ring having two free ends and which does provide a closed ring shape. Attachment 200 includes eyelets 202 at each end that are used to secure the attachment 200 to the two ends of the ring or band, as described below. Attachment may be made by passing a single suture through the eyelets and the existing ring or band. The attachment 200 further includes tines/hooks/barbs 204 to embed themselves in the annular tissue.

FIG. 10 illustrates attachment 200 bridging the gap between two ends 206/208 of the incomplete ring or band 210 at the mitral valve. The orientation of the attachment 200 and the ring 210 relative to the anterior mitral leaflet (AML) and posterior mitral leaflet (PML) is shown. The attachment 200 is tied in place concurrently with the implantation of the incomplete ring/band 210 or afterwards. The length of the attachment 200 and shape could be predetermined to fit a particular type and size of the ring as the gap between the ends of incomplete ring 202 is known. Ring 210 is secured to the annular tissue via sutures 212.

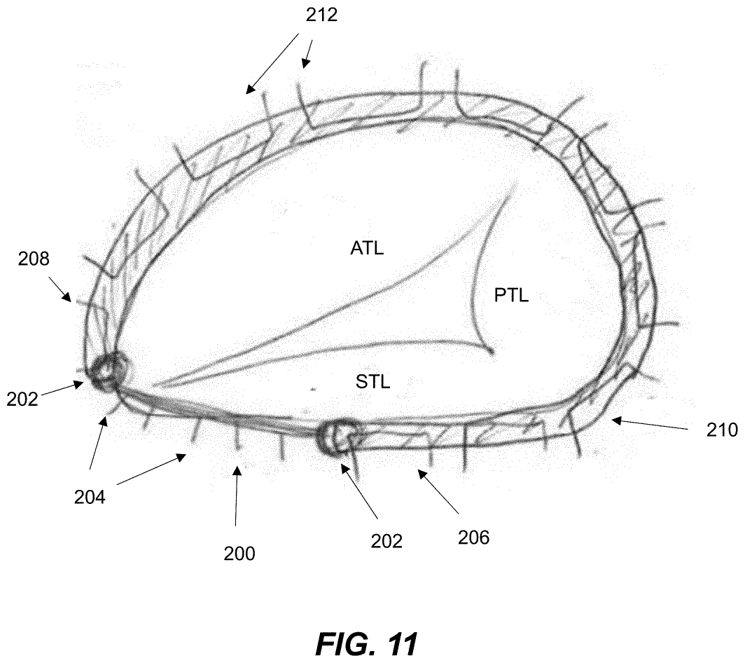

FIG. 11 illustrates attachment 200 bridging the gap between two ends 206/208 of the incomplete ring or band 210 at the tricuspid valve. The orientation of the attachment 200 and the ring 210 relative to the anterior tricuspid leaflet (ATL), the septal tricuspid leaflet (STL) and posterior tricuspid leaflet (PTL) is shown. The attachment 200 is secured to the ring 200 in the same manner as described above.

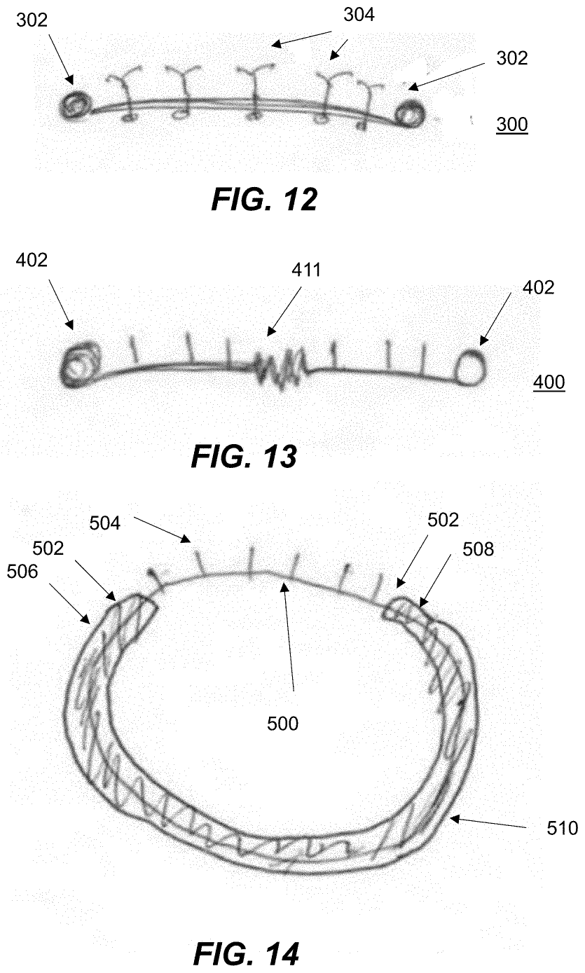

The attachment may not need additional suturing as it is held in place and is engaged with the annulus (anterior annulus in case of the Mitral valve and the open space in case of Tricuspid valve) with help of anchors. The anchors could be barbs of varying length and direction, tines, or anchors. FIG. 12 illustrates an embodiment of attachment 300 in which hooks 304 are driven though the attachment and into tissue.

FIG. 13 illustrates an embodiment of attachment 400 with an expandable portion 411 that can be used to adjust the length of the attachment 400. The expandable portion, which can be a coiled spring, allows for the attachment 400 to be elongated if and when needed.

As illustrated in FIG. 14, the existing band 510 can include a pre-attached anterior element 500. The element 500 resembles the attachment 200, 300, 400 described above. Such attachment portion 500 is preattached to the ring/band 510, such that the ends 502 of the attachment 500 are secured to the ends 506/508 of the ring/band 510. After suturing the ring/band 510 posteriorly, the anterior element 500 can be pushed in to the anterior annulus and secured thereto by use of the tines/barbs/anchors 504 as discussed above.

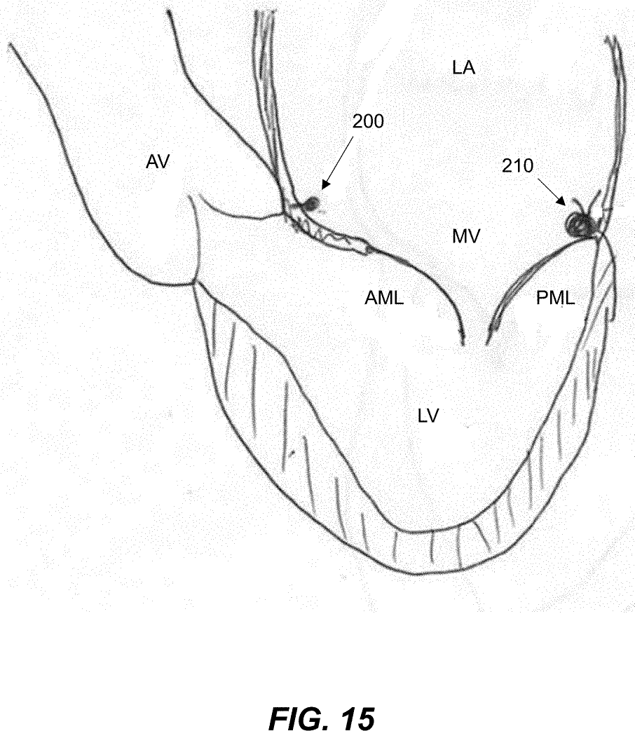

With time the attachment will get embedded in the tissue with time and will allow extra support to the anterior annulus during a Valve-in-ring procedure in future. FIG. 15 is a cross-section of the heart, which illustrates the relationship of the attachment and the incomplete ring/band in connection to the mitral valve (MV) including the anterior mitral leaflet (AML) and posterior mitral leaflet (PML). FIG. 15 illustrates that attachment 200 is embedded in the anterior annulus, and the incomplete ring/band 210 is sutured to the posterior annulus.

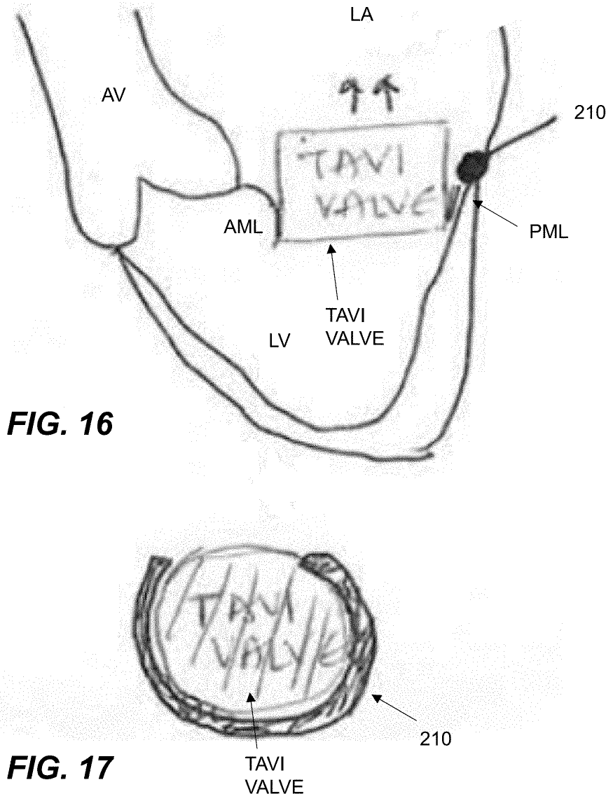

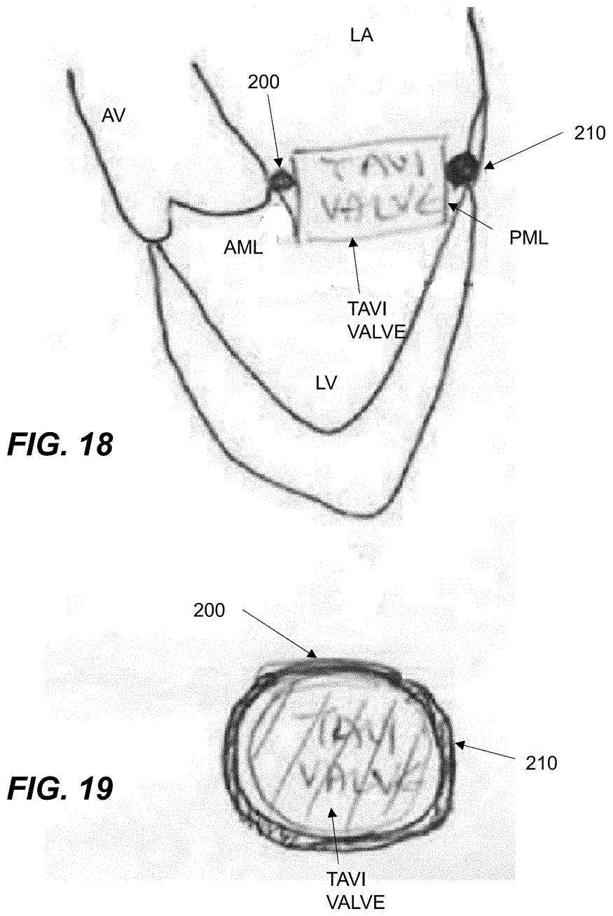

FIGS. 16 and 17 illustrate a heart that is not suitable for transcatheter aortic valve implantation (TAVI) because there is no anterior anchoring of the band/ring 210. FIGS. 18 and 19 illustrate a heart that is more suitable for TAVI due to the anterior anchoring of the band/ring 210 by use of attachment 200 providing a stable base for the valve.

It is understood that the subject matter described herein is not limited to particular embodiments described, as such may, of course, vary. For example, the exemplary embodiments describe above are not limited to fine needle aspiration applications. Instead the disclosed subject matter is applicable to additional clinical settings such as processing small surgical biopsies (less than 2 cm), in research laboratories for isolating cells from bone marrow diluted by blood, analyzing small samples of engineered tissues, and purifying cells in a spin column. Accordingly, nothing contained in the Abstract or the Summary should be understood as limiting the scope of the disclosure. It is also understood that the terminology used herein is for the purpose of describing particular embodiments only, and is not intended to be limiting. Where a range of values is provided, it is understood that each intervening value between the upper and lower limit of that range and any other stated or intervening value in that stated range, is encompassed within the disclosed subject matter.

What is claimed is:

1. An annuloplasty ring comprising:
a core defining a closed ring and comprising one or more flexible portions, wherein the core is capable of deformation about the flexible portion between a first configuration and a second configuration upon application of a predetermined force;
a resilient intermediate layer; and
a fabric cover layer.

2. The annuloplasty ring of claim 1, wherein the flexible portions of the core comprise polymer.

3. The annuloplasty ring of claim 1, wherein the flexible portions of the core comprise a metallic coil.

4. The annuloplasty ring of claim 1, wherein the core comprises a plurality of metal wires.

5. The annuloplasty ring of claim 4, wherein the wires are fabricated of Elgiloy or Nitinol.

6. The annuloplasty ring of claim 1, wherein the core comprises titanium.

7. The annuloplasty ring of claim 1, wherein the core comprises PEEK.

8. The annuloplasty ring of claim 1, wherein the intermediate layer comprises polymer or rubber.

9. An annuloplasty ring comprising:
a core defining a closed ring and comprising one or more weakened portions, wherein breakage of the weakened portions upon application of a predetermined force causes deformation between a first configuration and a second configuration;
a resilient intermediate layer; and
a fabric cover layer.

10. The annuloplasty ring of claim 9, wherein the weakened portions of the core comprise a smaller dimension than adjacent portions of the core.

11. The annuloplasty ring of claim 9, wherein the weakened portions of the core define a plurality of perforations therethrough.

12. The annuloplasty ring of claim 9, wherein the weakened portions of the core comprise a more brittle material than adjacent portions of the core.

13. The annuloplasty ring of claim 9, wherein the weakened portions of the core comprise a softer material than adjacent portions of the core.

14. The annuloplasty ring of claim 9, wherein the core comprises a plurality of metal wires.

15. The annuloplasty ring of claim 14, wherein the wires are fabricated of Elgiloy or Nitinol.

16. The annuloplasty ring of claim 9, wherein the core comprises titanium.

17. The annuloplasty ring of claim 9, wherein the core comprises PEEK.

18. An annuloplasty attachment for use with an annuloplasty ring comprising:
a body portion defining end portions and a plurality of engagement members for securement to the annular tissue; and
connection members disposed at each end portions of the body portion for connection with the end portions of an incomplete annuloplasty ring.

19. The annuloplasty attachment of claim 18, wherein the connection members are eyelets configured for suturing to the end portions of the incomplete annuloplasty ring.

20. The annuloplasty attachment of claim 18, wherein the engagement members are barbs, tines or anchors.
